(19) United States
(12) Patent Application Publication
(10) Pub. No.: US 2024/0216673 A1
(43) Pub. Date: Jul. 4, 2024

(54) DEVICE FOR CONNECTING AN IMPLANTABLE HEART PROSTHESIS TO THE VASCULAR SYSTEM OF A PATIENT, AND HEART PROSTHESIS PROVIDED WITH SUCH A CONNECTING DEVICE

(71) Applicant: CARMAT, Vélizy-Villacoublay (FR)

(72) Inventors: Foulques PARRA D'ANDERT, Gif-sur-Yvette (FR); Marc GRIMMÉ, Palaiseau (FR); Pascal GOUMAULT, Riom (FR); Pascal DUBUIS, Clerlande (FR)

(21) Appl. No.: 18/285,605

(22) PCT Filed: Mar. 8, 2022
(86) PCT No.: PCT/FR2022/050413

§ 371 Date: Oct. 4, 2023

(30) Foreign Application Priority Data
Apr. 6, 2021 (FR) 2103482

(51) Int. Cl. A61M 60/859; A61M 60/196

(52) U.S. Cl. CPC A61M 60/859; A61M 60/196

(57) ABSTRACT

A device for connecting an implantable heart prosthesis to the vascular system of a patient. The connecting device, intended for connecting a heart prosthesis, implantable in the pericardial cavity of a patient, to the vascular system of the patient, has an interface component equipped with a mitral interface element, a tricuspid interface element, an aortic interface element and a pulmonary interface element, which are intended for connection to the left atrium, the right atrium, the aorta and the pulmonary artery of the patient, each of the interface elements being provided with an orifice, and at least some of the interface elements having different orientations appropriate for taking up the orientations of the heart prosthesis and for respecting the anatomy of the patient, this connecting device making it possible in particular to fit the heart prosthesis in place more easily and more quickly.

### TECHNICAL FIELD

The present invention relates to a connecting device for connecting a heart prosthesis to the vascular system of a patient, said heart prosthesis being implantable in the pericardial cavity of the patient and being able to replace the natural left and right ventricles of said patient after their ablation. The present invention also relates to a heart prosthesis equipped with such a connecting device.

### PRIOR ART

A fully implantable heart prosthesis, as described for example in the patents FR-2 784 585 and FR-2 902 345, has the function of replacing the ventricles of the patient while retaining the atria. To achieve this, it comprises a rigid prosthesis body in which artificial ventricles are arranged.

To implant this heart prosthesis in the pericardial cavity of the patient, it is therefore necessary to create interfaces between the vascular system (atria and arteries) of the patient and the heart prosthesis while respecting the anatomy of the patient. To achieve this, we need to take into account:
a defined orientation of the atria (left and right) with a more or less constant spacing; and
the orientation and an arrangement of the arteries (aorta and pulmonary artery), which can vary considerably.

One solution currently in use comprises, on the one hand, an interface part (corresponding to a bezel system) for the admissions of the heart prosthesis and, on the other hand, two flexible conduits (or vascular conduits) for the ejections of the heart prosthesis.

The patents FR-2 902 343 and FR-2 902 344 describe a system of cooperating bezels, as an admission interface, for the admission of a heart prosthesis. This bezel system comprises a first bezel forming an integral portion of the rigid prosthesis body of the heart prosthesis and comprising first and second orifices communicating respectively with the left artificial ventricle and with the right artificial ventricle of the heart prosthesis, and a second bezel comprising third and fourth orifices able to be connected respectively to the left natural atrium and to the right natural atrium of the patient via collars which are sutured to the atria. These first and second bezels are configured so that they can be removably connected to each other, with the first and third orifices facing each other and the second and fourth orifices facing each other. This bezel system makes it easier to connect the heart prosthesis (fitted with the first bezel) to the second bezel (previously connected to the natural atria).

Each of the bezels in this admission interface is carried out before the heart prosthesis is placed. In contrast, the vascular conduits are first fitted to the heart prosthesis using staples, before being sutured to the arteries of the patient. The presence of the heart prosthesis means that the surgeon has less room to operate and thus a more difficult access to carry out the sutures. The final placement of the heart prosthesis is carried out by clipping the heart prosthesis onto the admission interface.

The presence of the heart prosthesis when the vascular conduits are sutured to the arteries poses an ergonomic problem. The heart prosthesis must be held in place by someone other than the surgeon carrying out the sutures, and the access to the arteries is limited. This means that two people are needed to place it.

In addition, the system for attaching the vascular conduits requires the use of several implantation ancillaries, and assembly is difficult (tight fitting) due to the sealing requirements.

### DESCRIPTION OF THE INVENTION

The purpose of this invention is to find a solution that allows to simplify and improve the implantation operation for the heart prosthesis by allowing the surgeon to work in a less constrained environment for the placement and the sutures. For this purpose, it relates to a connecting device intended for connecting a heart prosthesis implantable in the pericardial cavity of a patient to the vascular system of the patient, said heart prosthesis being capable of replacing the natural left and right ventricles of the patient after ablation of the latter.

According to the invention, said connecting device comprises a single interface part configured so that it can be attached to the heart prosthesis, the interface part being equipped with an interface element referred to as mitral interface element, an interface element referred to as tricuspid interface element, an interface element referred to as aortic interface element and an interface element referred to as pulmonary interface element intended, respectively, for the connection to the left atrium, to the right atrium, to the aorta and to the pulmonary artery of the patient, each of said interface elements being provided with an orifice, and said interface elements having predetermined (appropriate) orientations.

Thus, thanks to the invention, the connecting device provided with a single interface part comprising the assembly of the interface elements with, moreover, appropriate orientations of these interface elements as specified below, allows to simplify and facilitate the operation of placing the heart prosthesis by allowing the surgeon to work in a less constrained environment for the placement and the suture operations.

As will also be explained below, said connecting device offers many other advantages, including the following:
an improved ergonomics for the surgeon;
a reduction in the number of parts implanted and in the number of parts required for the implantation; and
a shorter implantation times.

In the context of the present invention, the relative orientations of the interface elements of the interface part are adapted both to the characteristics of the heart prosthesis and to a given anatomy, in particular an average anatomy, of patients suitable for receiving the heart prosthesis, the heart prosthesis being designed to respect this anatomy.

In addition, the relative positions (and distances) of the interface elements of the interface part are also adapted both to the characteristics of the heart prosthesis and to a given anatomy, in particular an average anatomy, of patients suitable for receiving the heart prosthesis.

Thus, advantageously, the orientation of the mitral interface element and the orientation of the tricuspid interface element have an angle between them of between 0° and 32°, and preferably between 0° and 20°. Furthermore, in a particular embodiment, the mitral interface element and the tricuspid interface element are arranged on a flat plate of the interface part, which notably allows to facilitate the realization.

In addition, advantageously, the interface part has at least some of the following orientations:
the orientation of the aortic interface element and the orientation of the mitral interface element have, between them, an angle of between 0° and 90°, and preferably between 20° and 45°;
the orientation of the aortic interface element and the orientation of the tricuspid interface element have, between them, an angle of between 0° and 90°, and preferably between 20° and 45°;
the orientation of the pulmonary interface element and the orientation of the mitral interface element have, between them, an angle of between 26° and 64°, and preferably between 40° and 60°;
the orientation of the pulmonary interface element and the orientation of the tricuspid interface element have, between them, an angle of between 31° and 73°, and preferably between 40° and 60°;
the orientation of the aortic interface element and the orientation of the pulmonary interface element have, between them, an angle of between 39° and 79°, and preferably between 55° and 70°.

In addition, advantageously, the interface part has at least some of the following distances:
the distance between the centre of the orifice of the mitral interface element and the centre of the orifice of the tricuspid interface element is between 42 mm and 76 mm, and preferably between 45 mm and 60 mm;
the distance between the centre of the orifice of the mitral interface element and the centre of the orifice of the aortic interface element is between 37 mm and 65 mm, and preferably between 40 mm and 55 mm;
the distance between the centre of the orifice of the mitral interface element and the centre of the orifice of the pulmonary interface element is between 50 mm and 80 mm, and preferably between 60 mm and 70 mm;
the distance between the centre of the orifice of the tricuspid interface element and the centre of the orifice of the aortic interface element is between 34 mm and 64 mm, and preferably between 35 mm and 50 mm;
the distance between the centre of the orifice of the tricuspid interface element and the centre of the orifice of the pulmonary interface element is between 59 mm and 91 mm, and preferably between 70 mm and 80 mm;
the distance between the centre of the orifice of the aortic interface element and the centre of the orifice of the pulmonary interface element is between 22 mm and 46 mm, and preferably between 30 mm and 40 mm.

In a preferred embodiment, the interface part is made from one of the following materials: titanium or stainless steel.

In addition, advantageously, the aortic interface element and the pulmonary interface element each comprise a thread, to allow a screwing, as specified below.

In a preferred embodiment, the connecting device also comprises:
a first vascular conduit, a first end of which is intended to be sutured to the aorta and the second end of which is equipped with a ring mounted so as to rotate freely and capable of being screwed onto the aortic interface element; and/or
a second vascular conduit, a first end of which is intended to be sutured to the pulmonary artery and the second end of which is equipped with a ring mounted so as to rotate freely and capable of being screwed onto the pulmonary interface element; and/or
a first suture collar intended to be sutured to the left atrium and comprising a core configured so that it can be mounted on the mitral interface element; and/or
a second suture collar intended to be sutured to the right atrium of the patient and comprising a core configured so that it can be mounted on the tricuspid interface element.

The present invention also relates to a heart prosthesis implantable in the pericardial cavity of a patient, said heart prosthesis being able to replace the natural left and right ventricles of the patient after ablation of the latter.

According to the invention, the heart prosthesis comprises at least one prosthesis body, in which at least left and right artificial ventricles are arranged, as well as a connecting device such as that described above, which is configured so that it can be attached in a housing provided in the prosthesis body.

Advantageously, the interface part of the connecting device comprises a centring tongue which cooperates with a gorge fitted in the external wall of the prosthesis body, to make it easier to place the prosthesis on the connecting device.

Once in place in the housing, the connecting device is attached to the prosthesis body. Within the scope of the present invention, various attachment means are conceivable for removably attaching the interface part to the prosthesis body.

In a first embodiment, the heart prosthesis comprises screw attachment means, while in a second embodiment, the heart prosthesis comprises clip-attachment means.

### BRIEF DESCRIPTION OF FIGURES

The attached figures will make it clear how the invention can be carried out. In these figures, identical references designate similar elements.

FIG. 1 is a perspective view of an external face of an interface part of a connecting device.

FIG. 2 is a schematic perspective view of a part of a heart prosthesis provided with a connecting device.

FIG. 3 is a similar figure to FIG. 1 showing different orientation and distance characteristics of interface elements of the interface part.

FIG. 4 is a perspective view of an internal face of an interface part of a connecting device, provided with vascular conduits that are partially represented.

FIG. 5 is a perspective view of the internal face of the interface part in FIG. 1, showing different planes of support.

FIG. 6 is a schematic perspective view of a part of a prosthesis body of a heart prosthesis, provided with a housing intended to receive a connecting device.

FIG. 7 is a perspective view of a vascular conduit of a connecting device.

FIG. 8 is a perspective view of a suture collar of a connecting device.

FIG. 9 is a schematic perspective view of screw attachment means.

FIG. 10 is a schematic lateral view of clip attachment means.

### DETAILED DESCRIPTION

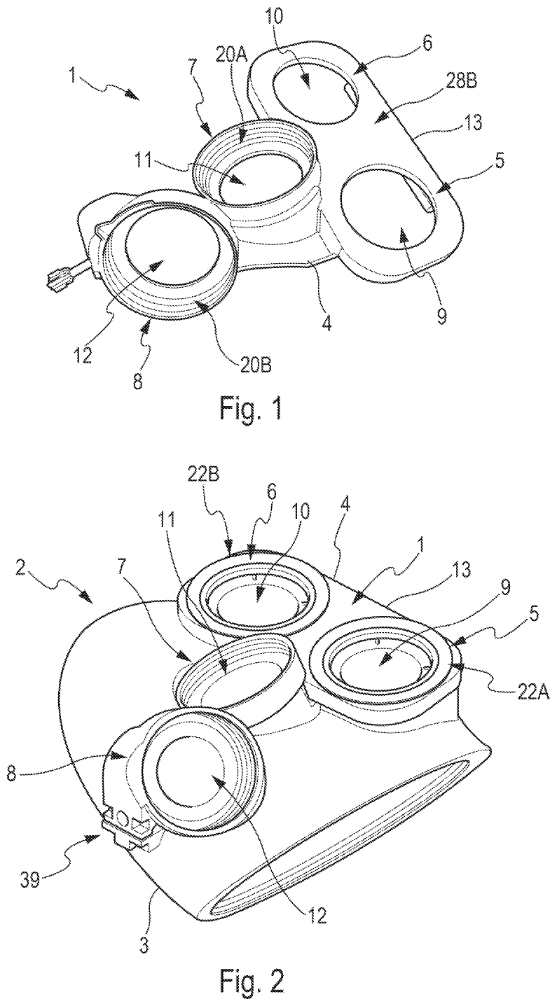

The connecting device 1 illustrating the invention and shown schematically in a particular embodiment in FIG. 1 is designed to connect a heart prosthesis 2 to the vascular system (not shown) of a patient.

This heart prosthesis 2, shown in part in FIG. 2, can be implanted in the thoracic and pericardial cavity of the patient and is capable of replacing his natural left and right ventricles after their ablation.

To achieve this, the heart prosthesis 2 comprises a rigid prosthesis body 3, in which artificial left and right ventricles (not shown) are intended to replace the natural left and right ventricles of the patient.

The heart prosthesis 2 comprises all the means necessary for its operation. In particular, it may comprise at least some of the characteristics presented in the aforementioned patents FR-2 784 585 and FR-2 902 345. The heart prosthesis 2, and in particular the elements arranged in the prosthesis body 3, are not described further in the following description.

The connecting device 1 is intended to connect the heart prosthesis 2 to the left (natural) atrium, to the right (natural) atrium, to the aorta (natural) and to the pulmonary artery (natural) of the patient.

To do this, said connecting device 1 comprises, according to the invention, as shown in FIG. 1, a (single) interface part 4 configured so that it can be attached to the prosthesis body 3 of the heart prosthesis 2.

This interface part 4 is a rigid, indented part. It follows the orientation of the heart prosthesis 2 and respects the anatomy of the patient, as described below.

To this end, the interface part 4 is provided with four interface elements, as shown in FIG. 1 (showing an external face 28B of the interface part 4):

an interface element referred to as mitral interface element 5, intended to connect the heart prosthesis to the left (natural) atrium of the patient;
an interface element referred to as tricuspid interface element 6, intended to connect the heart prosthesis to the right (natural) atrium of the patient;
an interface element referred to as aortic interface element 7, intended to connect the heart prosthesis to the (natural) aorta of the patient; and
an interface element referred to as pulmonary interface element 8, intended to connect the heart prosthesis to the pulmonary artery (natural) of the patient.

As a result, the single interface part 4 allows to carry out the connection with the heart prosthesis 2 for the assembly of its four accesses (two for admission and two for ejection) illustrated by openings 9A to 12A in FIG. 6, to access the artificial left and right ventricles of the heart prosthesis 2.

Furthermore, according to the invention, each of said interface elements 5 to 8 is provided with an orifice 9 to 12, and at least some of said interface elements 5 to 8 have different orientations, as specified below.

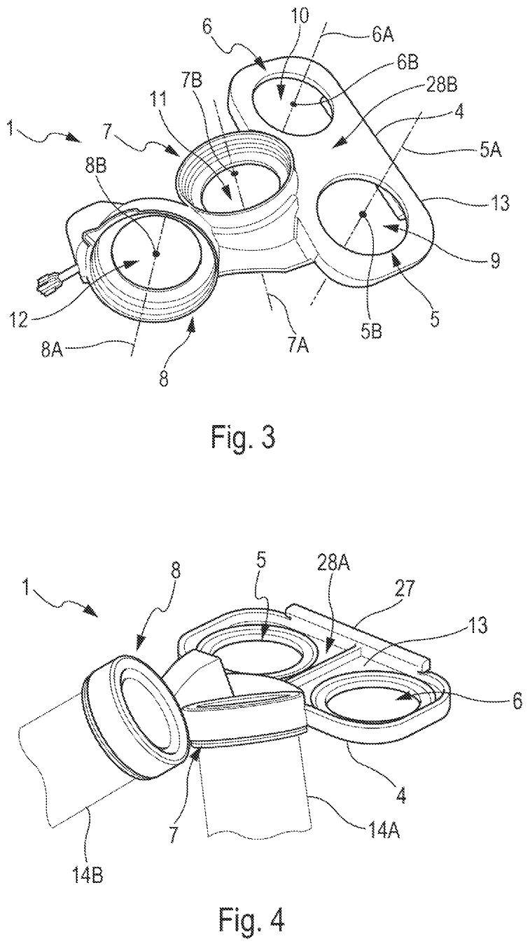

By “orientation” we mean the direction of the axis of the orifice of the interface element, as represented in FIG. 3 by axes 5A, 6A, 7A and 8A respectively for the orifices 9, 10, 11 and 12 of the interface elements 5, 6, 7 and 8.

The orientations can also be defined by the interface planes or support planes P1, P2, P3 and P4 specified below, which are orthogonal to said axes 5A, 6A, 7A and 8A.

The orientations of the interface elements 5 to 8 are adapted both to the technical characteristics of the heart prosthesis 2 and to the average anatomy of the potential patients, in particular to simplify the placement of the heart prosthesis 2.

In the particular embodiment shown, particularly in FIG. 3, the mitral interface element 5 and the tricuspid interface element 6 have the same orientation. The axes 5A and 6A are parallel. In addition, the mitral interface element 5 and the tricuspid interface element 6 are formed in a single and same flat plate 13 of the interface part 4, which will be positioned in the auricular plane of the patient.

However, in the context of the present invention, the orientation 5A of the mitral interface element 5 and the orientation 6A of the tricuspid interface element 6 may have a non-zero angle between them. More generally, they can have an angle between them of between 0° and 32°, and preferably between 0° and 20°.

In addition, the orientation 7A of the aortic interface element 7 and the orientation 5A of the mitral interface element 5 have an angle between them of between 0° and 90°, and preferably between 20° and 45°.

Similarly, the orientation 7A of the aortic interface element 7 and the orientation 6A of the tricuspid interface element 6 have, between them, an angle of between 0° and 90°, and preferably between 20° and 45°.

In addition, the orientation 8A of the pulmonary interface element 8 and the orientation 5A of the mitral interface element 5 have an angle between them of between 26° and 64°, and preferably between 40° and 60°.

Similarly, the orientation 8A of the pulmonary interface element 8 and the orientation 6A of the tricuspid interface element 6 have an angle between them of between 31° and 73°, and preferably between 40° and 60°.

Furthermore, the orientation 7A of the aortic interface element 7 and the orientation 8A of the pulmonary interface element 8 have, between them, an angle of between 39° and 79°, and preferably between 55° and 70°.

The orientations of the orifices (or interface planes) are defined by the design of the heart prosthesis 2 and are derived from patient physiological data.

Furthermore, by way of illustration, in the particular example shown in FIG. 3, the following distances can be provided between the centres 5B to 8B of the various orifices 9 to 12 of the interface part 4:
between 42 mm and 76 mm and preferably between 45 mm and 60 mm, for the distance between the centre 5B of the orifice 9 of the mitral interface element 5 and the centre 6B of the orifice 10 of the tricuspid interface element 6;
between 37 mm and 65 mm and preferably between 40 mm and 55 mm, for the distance between the centre 5B of the orifice 9 of the mitral interface element 5 and the centre 7B of the orifice 11 of the aortic interface element 7;
between 50 mm and 80 mm and preferably between 60 mm and 70 mm, for the distance between the centre 5B of the orifice 9 of the mitral interface element 5 and the centre 8B of the orifice 12 of the pulmonary interface element 8;
between 34 mm and 64 mm and preferably between 35 mm and 50 mm, for the distance between the centre 6B of the orifice 10 of the tricuspid interface element 6 and the centre 7B of the orifice 11 of the aortic interface element 7;
between 59 mm and 91 mm and preferably between 70 mm and 80 mm, for the distance between the centre 6B of the orifice 10 of the tricuspid interface element 6 and the centre 8B of the orifice 12 of the pulmonary interface element 8; and
between 22 mm and 46 mm and preferably between 30 mm and 40 mm, for the distance between the centre 7B of the orifice of the aortic interface element 7 and the centre 8B of the orifice 12 of the pulmonary interface element 8.

In a preferred embodiment, the distances and the angles between the interface elements 5 to 8 of the interface part 4 are defined on the basis of patient scans. As each patient has specific physiological and anatomical characteristics, any spacing are compensated by flexible parts (suture collars and vascular conduits) described below, which make the connection between the vascular system of the patient and the connecting device 1.

In a particular embodiment, the size of the interface part 4 is such that it can be inscribed, in a plan view, in a rectangle of 12 centimetres long and 9 centimetres wide.

The material used to manufacture the interface part 4 must be sufficiently rigid to withstand the attachment forces. In particular, a metallic material such as stainless steel 316L can be used.

In a preferred embodiment, the interface part 4 is made of titanium. Titanium is a biocompatible material with advantageous properties in terms of mass and mechanical strength. In this way, it is possible to produce an interface part 4 that is light but strong enough to withstand the forces of attachment of the vascular conduits (specified below) and of the heart prosthesis 2. Once the heart prosthesis 2 is in place, the resulting stresses are limited.

Preference is given to the pure titanium T40, which has good mechanical properties and an excellent biocompatibility, with a lower density than stainless steel (the mass of the connecting device 1 needs to be limited for the patient).

In a preferred embodiment, the connecting device 1 comprises a vascular conduit 14A which cooperates with the aortic interface element 7 of the interface part 4 and a vascular conduit 14B which cooperates with the pulmonary interface element 8 of the interface part 4, as shown in FIG. 4.

Preferably, the vascular conduits 14A and 14B are made in a similar way.

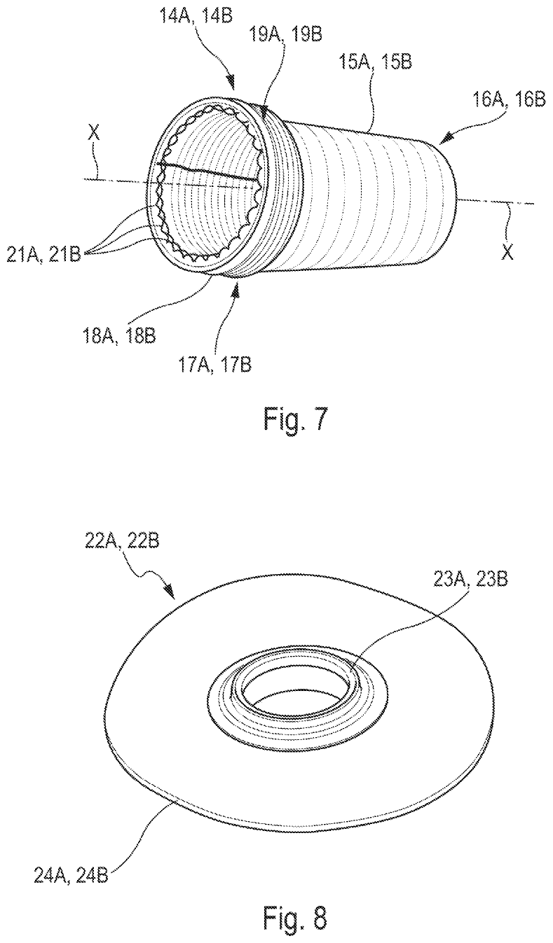

In a preferred embodiment shown in FIG. 7, the vascular conduit 14A comprises a tubular element 15A made of fabric, of longitudinal axis X-X, and has a first end 16A which is intended to be sutured to the aorta (not shown) and a second end 17A which is equipped with a ring referred to as loose ring 18A, i.e. which is mounted so as to rotate freely. The vascular conduit 14A is intended to be screwed, by means of this loose ring 18A, onto the aortic interface element 7. Thanks to this loose ring 18A, the vascular conduit 14A can be screwed onto the aortic interface element 7 while maintaining its (correct) position during the screwing operation. The aortic interface element 7 comprises an internal thread 20A as shown in FIG. 1, to allow the ring 18A (provided with an external thread 19A) to be screwed on and thus make the connection of the end 17A of the vascular conduit 14A to the interface part 4.

Similarly, the vascular conduit 14B comprises a tubular element 15B made of fabric, of longitudinal axis X-X, and has, as shown in FIG. 7, a first end 16B which is intended to be sutured to the pulmonary artery (not shown) and a second end 17B which is equipped with a ring referred to as loose ring 18B, i.e. which is mounted so that it can rotate freely. The vascular conduit 14B is intended to be screwed, by means of this loose ring 18B, onto the pulmonary interface element 8. Thanks to this loose ring 18B, the vascular conduit 14B can be screwed onto the aortic interface element 8 while maintaining its (correct) position during the screwing operation. The pulmonary interface element 8 comprises an internal thread 20B as shown in FIG. 1, to allow the ring 18B (provided with an external thread 19B) to be screwed on and thus make the connection of the end 17B of the vascular conduit 14B to the interface part 4.

In a preferred embodiment, the ring 18A, 18B is made of titanium. It is sutured to the tubular element 15A, 15B of the vascular conduit 14A, 14B, via sutures 21A, 21B shown schematically in FIG. 7. Preferably, therefore, a screw attachment system is provided for the vascular conduits 14A and 14B, which is adapted to the connecting device 1. Within the scope of the present invention, however, an attachment system other than a screw attachment may also be envisaged.

During the implantation, the tubular fabric element 15A, 15B of the vascular conduit 14A, 14B is cut to the appropriate length (along the longitudinal axis X-X) so that the connecting device 1 can be connected to the aorta or to the pulmonary artery of the patient. As the tissue is flexible, the surgeon can also use suturing techniques to adapt its orientation and avoid a “kink” phenomenon.

The connecting device 1 also comprises a suture collar 22A intended to be sutured to the left atrium of the patient and a suture collar 22B intended to be sutured to the right atrium of the patient.

Preferably, the suture collars 22A, 22B are made in a similar way.

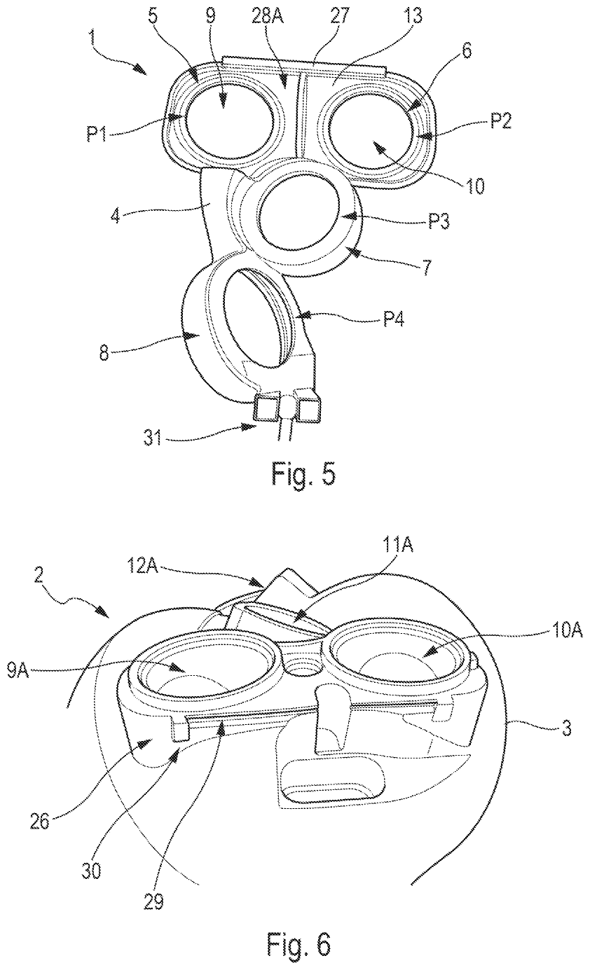

Each of said suture collars 22A, 22B comprises, as shown in FIG. 8, a core 23A, 23B, preferably made of silicone, which is configured so that it can be nested onto the mitral interface element or the tricuspid interface element. More specifically, when the suture collar 22A, 22B is placed, the core 23A, 23B is clamped between the heart prosthesis 2 and the interface part 4. To this end, the core 23A is supported on the support plane P1 shown in FIG. 5, which is provided on the internal face 28A of the interface part 4, around the orifice 9. Similarly, the core 23B is supported on the support plane P2 which is also provided on the internal face 28A of the interface part 4, around the orifice 10.

Each of said suture collars 22A, 22B comprises, in addition to the silicone core 23A, 23B, an interface surface 24A, 24B intended to be sutured to the tissues of the patient (atria).

The suture collars 22A, 22B are therefore used to connect the atria (left and right) of the patient to the input elements of the heart prosthesis 2, via the mitral and tricuspid interface elements.

The connecting device 1, as described above, is configured so that it can be installed in a housing 26 provided in the prosthesis body 3 of the heart prosthesis 2, as shown in FIG. 6. The shape of the housing 26 is adapted and complementary to the shape of the internal face 28A (FIG. 5) of the interface part 4. In particular, the bearing planes P3 and P4 of the mitral interface element 5 and the tricuspid interface element 6 respectively come into contact with corresponding areas of the housing 26. As shown in FIG. 6, the prosthesis body 3 comprises the openings 9A, 10A, 11A and 12A which are intended to be positioned opposite orifices 9, 10, 11 and 12 respectively of the interface part 4 when the latter is in place on the prosthesis body 3.

To make it easier to fit and hold, the interface part 4 of the connecting device 1 comprises a longitudinal centring tongue 27. As shown in FIGS. 4 and 5, this centring tongue 27 projects from the internal surface 28A of the flat plate 13 of the interface part 4, at one end of the flat plate 13. This centring tongue 27 is designed to be inserted into an elongated gorge 29 (FIG. 6) fitted in the external wall 30 of the prosthesis body 3 of the heart prosthesis 2, at the level of the housing 26.

In the context of the present invention, the connecting device 1 can be attached to the heart prosthesis 2 in various ways, after it has been placed in the housing 26, as shown in FIG. 2.

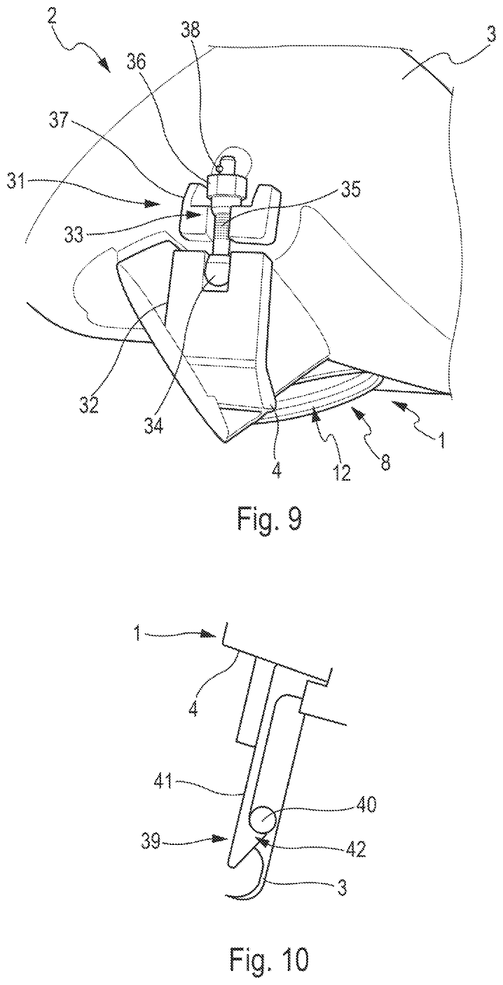

In a first embodiment, the heart prosthesis 2 comprises screw-attachment means 31 for removably attaching (by screwing) the interface part 4 to the prosthesis body 3. These attachment means 31 comprise, as shown in FIG. 9:
a leg 32 secured to the interface part 4, which is provided with a pin 33. This pin 33 is equipped at one of its ends with a head 34 movably mounted in a seat provided in the leg 32, and is provided with a thread 35 at the other (free) end, intended to receive a nut 36; and
a support plate 37 secured to the prosthesis body 3, intended to cooperate with the free end of the pin 33.

Once the connecting device 1 has been placed on the prosthesis body 3, the pin 33 is folded back onto the prosthesis body 3 and the integrated nut 36 is screwed on to hold it against the support plate 37, in order to lock the connecting device 1. A safety anchor 38 can be placed to prevent an unwanted unscrewing. Once the heart prosthesis 2 has been implanted, the area intended to be screwed down is visible and easily accessible to the surgeon.

In addition, in a second embodiment, the heart prosthesis 2 comprises clip-attachment means 39 for removably attaching (by clipping) the interface part 4 to the prosthesis body 3. These attachment means 39 comprise, as shown in FIG. 10, a pin 40 (or allowance on the prosthesis body 3) integrated into the heart prosthesis 2 and an attachment leg 41 secured to the interface part 4. This attachment leg 41 is provided with a hook 42 at its free end and is slightly deformable so that it can be hooked via the hook 42 with a slight force to the pin 40 and locked once the force is released.

The interface part 4, as described above, has the following advantageous characteristics:
it is biocompatible;
it can be implanted for a long time;
it can be cleaned and suitable to be sterilised;
it guarantees the sealing with the heart prosthesis 2; and
it is provided with an ergonomic and rapid attachment mode (which allows to reduce the duration of the extracorporeal circulation).

The interface part 4 is therefore intended to be connected:
on the one hand, to the atria (right and left) which are prepared beforehand (with the suture of the suture collars 22A and 22B), and to the vascular conduits 14A and 14B which are sutured to the arteries (aorta and pulmonary artery); and
on the other side, directly to the heart prosthesis 2 provided with the housing 26 in which the connecting device 1 fits, to complete the surface in contact with the tissues.

The main steps involved in the placement of the connecting device 1 and of the heart prosthesis 2 are described below.

After ablation of the left and right ventricles of the patient, the suture collars 22A and 22B are sutured to the left and right atria of the patient. The interface part 4 is then placed on the atria. The auricular plane forms a relatively rigid reference plane which will allow the interface part 4 (whose interface elements have the appropriate distances and orientations specified above) to position itself correctly in relation to the arteries.

When the interface part 4 is placed on the atria, the orientation of the vascular conduits 14A and 14B and the distance for cutting these vascular conduits 14A and 14B to the appropriate length are naturally defined. As the interface part 4 is adapted to the heart prosthesis 2 for a rapid attachment, the orientations of the heart prosthesis 2 are already respected and the sutures will not be stressed when it is placed.

Each of the vascular conduits 14A and 14B is then cut to the appropriate length.

Each of the vascular conduits 14A and 14B is screwed via its end 17A, 17B to the corresponding interface element 7, 8 of the interface part 4, with the aid of the ring 18A, 18B integrated into the vascular conduit 14A, 14B and is sutured via its other end 16A, 16B to the corresponding artery (aorta, pulmonary artery).

The vascular conduits 14A and 14B are therefore placed in the absence of the prosthesis body 3, which facilitates the work of the surgeon. Moreover, only one surgeon can carry out these operations.

Once the sutures have been performed and the connecting device 1 placed, the heart prosthesis 2 is mounted on the connecting device 1 and is attached to the latter. For mounting, the interface part 4 is placed in the housing 26 provided for this purpose in the external wall 30 of the prosthesis body. For the attachment, a screwing or a clipping are done, depending on the embodiment chosen for the attachment means, as described above.

This allows the heart prosthesis 2 to be attached to an interface part 4 (rigid) which respects the anatomy of the patient, via:
respecting the average spacing between the atria;
cutting vascular conduits to the correct length;
respecting the average orientation of the vascular conduits; and
carrying out the suture according to the reinforcement areas to be favoured (allowing to limit the “kink” phenomenon).

The interface part 4 therefore allows the patient to be prepared ergonomically before the heart prosthesis 2 is placed. In addition to saving time, this solution eliminates the need to use implantation ancillaries to place the vascular conduits 14A and 14B, as the connecting device 1 already meets this need. As a result, fewer parts have to be supplied, and therefore fewer parts are used in the operating theatre, which also generates cost savings. The interface part 4 thus serves both as an implantation ancillary and as element of the implanted heart prosthesis 2.

The connecting device 1 and the heart prosthesis 2, as described above, therefore have many advantages. In particular, they allow to obtain:
an improved ergonomics for the surgeon;
a reduction in the number of parts implanted (no staples in particular);
a reduction in the number of parts (or ancillaries) required for the implantation; and
a shorter implantation times.

1. A connecting device for connecting a heart prosthesis implantable in a pericardial cavity of a patient to a vascular system of the patient, wherein the heart prosthesis is capable of replacing natural left and right ventricles of the patient after ablation of the latter,
wherein the connecting device comprises a single interface part configured to be attached to the heart prosthesis, wherein the single interface part is equipped with an interface element referred to as mitral interface element, an interface element referred to as tricuspid interface element, an interface element referred to as aortic interface element and an interface element referred to as pulmonary interface element intended, respectively, for the connection to a left atrium, to a right atrium, to an aorta and to a pulmonary artery of the patient, each of the interface elements being provided with an orifice, and the interface elements having predetermined orientations.

2. The connecting device according to claim 1,
wherein an orientation of the mitral interface element and an orientation of the tricuspid interface element have an angle between them of between 0° and 32°.

3. The connecting device according to claim 1,
wherein the mitral interface element and the tricuspid interface element are arranged on a flat plate of the single interface part.

4. The connecting device according to claim 1,
wherein the single interface part has at least some of the following orientations:
the orientation of the aortic interface element of the mitral interface element have, between them, an angle of between 0° and 90°;
the orientation of the aortic interface element and the orientation of the tricuspid interface element have, between them, an angle of between 0° and 92°;
the orientation of the pulmonary interface element and the orientation (5A) of the mitral interface element have, between them, an angle of between 26° and 64°;
the orientation of the pulmonary interface element and the orientation (6A) of the tricuspid interface element have, between them, an angle of between 31° and 73°; and
the orientation of the aortic interface element and the orientation of the pulmonary interface element have, between them, an angle of between 39° and 79°.

5. The connecting device according to claim 1,
wherein the single interface part has at least some of the following distances:
the distance between a centre of the orifice of the mitral interface element and a centre of the orifice of the tricuspid interface element is between 42 mm and 76 mm;
the distance between the centre of the orifice of the aortic interface element is between 37 mm and 65 mm;
the distance between the centre of the orifice of the mitral interface element and the centre of the orifice is between 50 mm and 80 mm;
the distance between the centre of the orifice of the tricuspid interface element and the centre of the orifice of the aortic interface element is between 34 mm and 64 mm;
the distance between the centre of the orifice of the tricuspid interface element and the centre of the orifice of the pulmonary interface element is between 59 mm and 91 mm; and
the distance between the centre of the orifice of the aortic interface element and the centre of the orifice of the pulmonary interface element is between 22 mm and 46 mm.

6. The connecting device according to claim 1,
wherein the aortic interface element and the pulmonary interface element each comprise a thread.

7. The connecting device according to claim 1,
wherein the single interface part is made of one of the following materials: titanium, stainless steel.

8. The connecting device according to claim 1,
wherein the connecting device further comprises:
a first vascular conduit, a first end of which is intended to be sutured to the aorta of the patient and a second end of which is equipped with a ring mounted so as to rotate freely and able to be screwed onto the aortic interface element; and
a second vascular conduit, a first end of which is intended to be sutured to the pulmonary artery of the patient and the second end of which is equipped with a ring mounted so as to rotate freely and capable of being screwed onto the pulmonary interface element.

9. The connecting device according to claim 1,
wherein the connecting device further comprises:
a first suture collar intended to be sutured to the left atrium of the patient and comprising a core configured to be mounted on the mitral interface element; and
second suture collar intended to be sutured to the right atrium of the patient and comprising a core configured to be mounted on the tricuspid interface element.

10. A heart prosthesis implantable in a pericardial cavity of a patient, the heart prosthesis being capable of replacing natural left and right ventricles of the patient after ablation of the latter,
wherein the heart prosthesis implantable comprises at least one rigid prosthesis body in which at least left and right artificial ventricles are arranged, and a connecting device according to claim 1, wherein the connecting device is configured to be attached in a housing provided in the at least one rigid prosthesis body.

11. The heart prosthesis as claimed in claim 10,
wherein the single interface part of the connecting device comprises a centring tongue which cooperates with a gorge fitted in an external wall of the at least one rigid prosthesis body.

12. The heart prosthesis according to claim 10,
wherein the heart prosthesis comprises a screw attachment for removably attaching the single interface part to the at least one rigid prosthesis body.

13. The heart prosthesis according to claim 10,
wherein the heart prosthesis comprises a clip-attachment for removably attaching the single interface part to the at least one rigid prosthesis body.
